(19) United States
(12) Patent Application Publication
(10) Pub. No.: US 2026/0174484 A1
(43) Pub. Date: Jun. 25, 2026

(54) SYSTEM AND METHOD FOR CAVITY EXPANSION IN SURGICAL OPERATIONS

(71) Applicant: Northwestern Memorial Healthcare, Chicago, IL (US)

(72) Inventor: Ankit BHARAT, Chicago, IL (US)

(21) Appl. No.: 19/429,996

(22) Filed: Dec. 22, 2025

Related U.S. Application Data
(60) Provisional application No. 63/736,944, filed on Dec. 20, 2024.

(51) Int. Cl. A61B 17/80; A61B 17/00; A61B 17/56; A61B 17/68; A61B 17/88

(52) U.S. Cl. CPC A61B 17/8076; A61B 17/8802; A61B 2017/00969; A61B 2017/564; A61B 2017/568; A61B 2017/681

(57) ABSTRACT

A surgical method for expanding a thoracic cage during a surgical procedure includes cutting through a thoracic cage to create an upper thoracic cage portion and a lower thoracic cage portion, separating the first thoracic cage portion and the second thoracic cage portion to open the thoracic cage, and installing, between the first thoracic cage portion and the second thoracic cage portion, a device that holds the first thoracic cage portion and the second thoracic cage portion apart from each other to expand a thoracic cavity of the thoracic cage. The surgical method includes a post-surgical cavity volume of the thoracic cage that is larger relative to a pre-surgical cavity volume of the thoracic cage.

### CROSS-REFERENCE TO RELATED APPLICATION(S)

This application is related to and claims the benefit of priority from U.S. Provisional Application No. 63/736,944, titled “SYSTEM AND METHOD FOR CAVITY EXPANSION IN SURGICAL OPERATIONS,” filed on Dec. 20, 2024, the full disclosure of which is hereby incorporated by reference in its entirety for all purposes.

### BACKGROUND

### 1. Field of Disclosure

Embodiments of the present disclosure relate to systems and methods for expanding the cavity of a torso during surgical operations. In particular, embodiments of the present disclosure relate to thoracic cavity expansion constructs and modular implants to facilitate organ transplantation by enlarging a recipient thoracic cavity.

### 2. Description of Related Art

In the field of organ transplants, transplants within the chest can be constrained by limited intrathoracic space defined by the ribs, sternum, costal cartilage, and associated soft tissues. For example, in the case of lung transplants, which have the lowest utilization of all donor organs, differences in lung sizes due to factors such as donor height, donor gender, etc. may create difficulties with finding a donor with an appropriate lung size to match a recipient. Donor-recipient size mismatch can result in a usable donor lung being rejected or forced downsizing (e.g., wedge/lobar reduction). Furthermore, recipients can have reduced thoracic cavity volume due to fibrosis, scarring, contracture, or chronic lung disease.

Moreover, potential donors are typically male and young, with larger lungs, and it is hard to use the larger lungs in a transplant in a smaller person. It can even be difficult to fit lungs into a recipient when the lungs would be the proper size for the recipient, because of the restricted space that exists in the thoracic cavity. Additionally, those who are in need of a lung transplant often have scarring and fibrosis on the lungs, and because their lungs are less strong and less functional, their lungs can also be smaller than the lungs of a typical person of their size. For example, a person in need of a lung transplant may be 6 feet tall, but they may have lungs that resemble a person who is 5 feet, 4 inches, meaning their thoracic cavity may have shrunk.

Attempting to place an oversized donor organ into a restricted cavity can create compression of adjacent structures (including the heart and mediastinal vessels) and can impair graft function. Existing approaches frequently lack (i) a predictable way to quantify and correct the volumetric deficit and (ii) reliable strategies for skeletal fixation and soft-tissue closure once the thoracic cage is expanded.

### SUMMARY

Applicant recognized the problems noted above herein and conceived and developed embodiments of thoracic expansion systems and methods, according to the present disclosure, that increase a thoracic cavity volume by creating and maintaining a controlled separation between thoracic cage segments during a surgical operation.

In an embodiment, a thoracic cavity expansion device for installation in a thoracic cage during a surgical procedure includes a molded body having a bone cement and a mesh envelope substantially surrounding the bone cement, the mesh envelope constraining the bone cement to define a shape of the device. The device further includes one or more fasteners arranged to secure the molded body to an upper sternal portion and to a lower sternal portion of the thoracic cage. Additionally, the device spans a sternotomy gap between the upper sternal portion and the lower sternal portion and maintains a separation between the upper and lower sternal portions that increases a post-surgical cavity volume of the thoracic cage relative to a pre-surgical cavity volume.

In another embodiment, a surgical method for expanding a thoracic cage during a surgical procedure includes cutting through a thoracic cage to create an upper thoracic cage portion, a lower thoracic cage portion, an upper sternal portion, and a lower sternal portion, separating the first thoracic cage portion and the second thoracic cage portion to open the thoracic cage, installing, between the first thoracic cage portion and the second thoracic cage portion, a device that holds the first thoracic cage portion and the second thoracic cage portion apart from each other to expand a thoracic cavity of the thoracic cage, fastening one or more fixation plates to bone cement, the first sternal portion, and the second sternal portion with the one or more fasteners. The method also includes the device having bone cement positioned between the first sternal portion and the second sternal portion, one or more fixation plates, and one or more fasteners. Additionally, the method includes a post-surgical cavity volume of the thoracic cage that is larger relative to a pre-surgical cavity volume of the thoracic cage.

### BRIEF DESCRIPTION OF DRAWINGS

The present technology will be better understood on reading the following detailed description of non-limiting embodiments thereof, and on examining the accompanying drawings, in which:

FIG. 1 is a perspective view of an embodiment of a device used to expand the thoracic cavity of a human body during an organ transplant surgical procedure, in accordance with embodiments of the present disclosure;

FIG. 2 is a perspective view of a human body with an embodiment of a device used to expand the thoracic cavity of a human body during an organ transplant surgical procedure, in accordance with embodiments of the present disclosure;

FIG. 3A is an example schematic cross-sectional view of a torso of a human body showing a thoracic cavity before installation of a device used to expand the thoracic cavity during an organ transplant surgical procedure, in accordance with embodiments of the present disclosure;

FIG. 3B is an example schematic cross-sectional view of a thoracic cavity of a human body showing a thoracic cavity expansion after installation of an embodiment of the device used to increase the volume of the thoracic cavity during an organ transplant surgical procedure, in accordance with embodiments of the present disclosure;

FIG. 4 is a flow chart of a surgical method for expanding the thoracic cavity of a human body during an organ transplant surgical procedure, in accordance with embodiments of the present disclosure;

FIG. 5 is a schematic view of a post-surgical system including a thoracic cavity expansion device positioned across a sternotomy gap in a torso, in accordance with embodiments of the present disclosure;

FIG. 6 is a schematic view of a thoracic expansion device comprising bone cement contained within mesh and secured by fixation plates, in accordance with embodiments of the present disclosure;

FIG. 7 is a flow chart of a method for thoracic cavity expansion during a surgical operation, in accordance with embodiments of the present disclosure;

FIG. 8 is an exploded schematic view of a modular thoracic expansion system with stackable spacer modules and a central retention rod, in accordance with embodiments of the present disclosure;

FIG. 9 is a schematic view of a soft-tissue bridging module captured between adjacent spacer modules, in accordance with embodiments of the present disclosure; and

FIG. 10 is a schematic view of a distal anchoring clamp which may be configured to engage a xiphoid process or costal margin, in accordance with embodiments of the present disclosure.

### DETAILED DESCRIPTION

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose. Additionally, like reference numerals may be used for like components, but such use should not be interpreted as limiting the disclosure.

When introducing elements of various embodiments of the present disclosure, the articles “a”, “an”, “the”, and “said” are intended to mean that there are one or more of the elements. The terms “comprising”, “including”, and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments”, or “other embodiments” of the present disclosure are not intended to be interpreted as excluding the existence of additional embodiments that also incorporate the recited features. Furthermore, reference to terms such as “above”, “below”, “upper”, “lower”, “side”, “front”, “back”, or other terms regarding orientation or direction are made with reference to the illustrated embodiments and are not intended to be limiting or exclude other orientations or directions. Moreover, references to “substantially” or “approximately” or “about” may refer to differences within ranges of +/−10 percent.

Furthermore, like numbers may be used to refer to like elements throughout, but it should be appreciated that using like numbers is for convenience and clarity and not intended to limit embodiments of the present disclosure. For example, one or more subsequent figures may share similar features with one or more prior figures and the similar features may be identified with like reference numerals for convenience purposes only and not to limit the scope of the present disclosure.

Embodiments of the present disclosure are directed to systems and methods to expand a thoracic cavity of a human body during an organ transplant. This system and method may be implemented with any type of transplant involving an organ of the torso, however, embodiments of the present disclosure may be particularly related to implementing the system and method for lung transplants. The systems and methods described herein may be applied to heart transplantation, combined cardiopulmonary transplantation, or other thoracic procedures requiring increased working space and/or increased postoperative cavity volume. Accordingly, while embodiments may be described with specific reference to lungs, it should be appreciated that such description is for non-limiting illustrative purposes.

The system and method discussed herein may include the installation of a device in a thorax, which may function to expand the thoracic cavity, or to retain an expansion of the thoracic cavity. The expanding of the thoracic cavity may ease the installation of transplanted lungs in the body of the recipient, and it may also allow larger lungs from a donor to be used in a smaller recipient. Additionally, there may be better long-term outcomes for the recipient, because the transplanted lungs are not too small in a recipient that is too large.

Embodiments of the present disclosure are also directed toward a device for expanding a cavity of a torso of a human body that may include bone cement positioned between a first sternal portion and a second sternal portion of a sternum of the torso, the bone cement creating an artificially enlarged sternum. In some embodiments, the artificially enlarged sternum expands the cavity of the torso. Expansion of the cavity of the torso may refer to expansion in a “vertical” direction (e.g., expansion with respect to the frontal plane, such as from a posterior area to an anterior area), expansion in a “horizontal” direction (e.g., expansion with respect to the transverse plane, such as from left to right), and/or expansion in an “axial” direction (e.g., expansion with respect to the frontal plane, such as from an interior area to a superior area). The device for expanding a cavity of the torso of the human body may also include mesh substantially surrounding the bone cement, one or more fixation plates, and one or more fasteners, such as screws or the like. The one or more fixation plates of the device may be attachable to the first sternal portion, the second sternal portion, and the bone cement via the one or more fasteners.

Additionally, embodiments of the present disclosure are directed toward a surgical method that may involve cutting through a sternum, ribs, and/or a torso of a human body, where the torso contains a cavity. The cutting of the torso may create a first torso portion, a second torso portion, a first sternal portion, and a second sternal portion. The surgical method may also involve opening the torso via separating the first torso portion and the second torso portion and expanding the cavity of the torso. Additionally, the surgical method may involve installing a device in the torso in which the device is positioned between the first torso portion and the second torso portion and holds the first torso portion and the second torso portion apart from each other. The device used in the surgical method may include bone cement positioned between the first sternal portion and the second sternal portion, one or more fixation plates, and one or more fasteners, wherein the one or more fixation plates are attachable to the first sternal portion, the second sternal portion, and the bone cement via the one or more fasteners.

In some embodiments of the surgical method, the surgical method may further include installing sutures in the torso to stabilize the ribs, the sutures positioned between the first torso portion and the second torso portion. In an embodiment of the surgical method, the surgical method may additionally include advancing the pectoralis muscle of the torso of the human body. In another embodiment of the surgical method, the surgical method may further include closing an opening of the thorax made during the cutting of the torso.

Systems and methods of the present disclosure may be associated with a load-bearing interposition construct spanning a surgically created thoracic cage gap (e.g., between sternal segments) to maintain a controlled distraction distance and increase thoracic cavity volume during/after transplant. In at least one embodiment, a modular, stackable expansion column (e.g., standardized-height modules captured by a central retention rod) provides fast, intraoperative, quantized distraction with strong resistance to shear/rotation. Additionally, one or more embodiments may incorporate predictive sizing (software-assisted and/or manual-template assisted) that converts donor/recipient mismatch into a selected distraction distance, module count, and/or pre-defined implant category. Systems and methods of the present disclosure may also include soft-tissue integration elements (e.g., a skirt/scaffold captured by the construct) that create tension-reducing closure options when pectoralis advancement is limited. Furthermore, embodiments may include fixation elements to secure the load-bearing interposition construct to one or more portions of a recipient, which may include one or more of plates/screws, cerclage/straps, clamps, adhesives, quick-release interfaces, and distal anchoring to xiphoid/costal margin or other distal anatomy. As discussed herein, infection-mitigation and re-exploration/bleeding-risk options may also be incorporated, which may include antibiotic-eluting surfaces/materials, low-biofilm surfaces, and temporary/removable expanders (including inflatable variants).

One or more embodiments of the present disclosure include thoracic expansion systems and methods that increase a thoracic cavity volume by creating and maintaining a controlled separation between thoracic cage segments (e.g., separated sternal segments) during a surgical operation. In some embodiments, a monolithic interposition spacer is formed from curable material (e.g., bone cement) contained within a mesh envelope and secured to the thoracic cage using fixation plates and fasteners. In one or more embodiments, a modular expansion system comprises a central retention member and a plurality of stackable spacer modules. The stack can be compressed into a rigid load-bearing column to establish a selected distraction height. In at least one embodiment, a predictive sizing workflow receives recipient and donor parameters and outputs a target distraction distance, a module count, and/or a selection among pre-defined implant categories. Embodiments of the present disclosure may be associated with a soft-tissue bridging module (e.g., a synthetic skirt or scaffold) that is integrated with the expansion construct to facilitate closure and to reduce tension when native flap mobility is limited. Furthermore, systems and methods may further include distal anchoring and/or screwless fixation mechanisms (including clamps for xiphoid/costal margin engagement) for improved stability in low bone density or distal bone insufficiency scenarios. Additionally, in at least one embodiment, temporary and/or removable expansion constructs (including inflatable expanders) facilitate management of bleeding-risk or re-exploration.

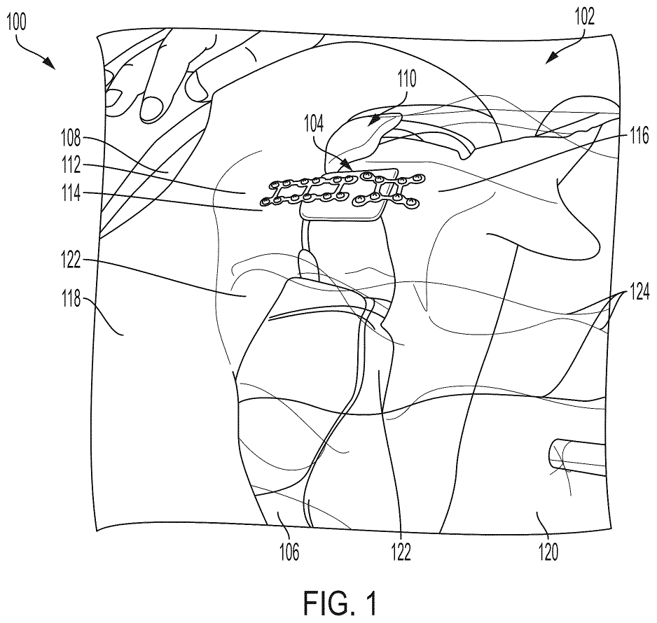

FIG. 1 is a perspective view of a human body 100 comprising a torso 102 (e.g., a thoracic cage 102) with an embodiment of an installed device 104 (e.g., a thoracic cavity expansion device 104, a thoracic expansion device 104, an expansion device 104, etc.) used to expand the thoracic cavity 106 of a thorax 108 of the human body 100 during an organ transplant surgical procedure. As should be appreciated, the torso 102 includes the thorax 108, as well as other body parts, such as the abdomen, pelvis, back, and others. FIG. 1 depicts the human body 100 with a horizontal sternotomy 110, the horizontal sternotomy 110 being substantially perpendicular to a sternum 112 of the human body 100. However, it should be appreciated that the sternotomy 110 may be a median sternotomy (e.g., substantially parallel to the sternum 112, or along the sternum 112) as well. In general, horizontal cuts may be more useful when performing a lung transplant, and median sternotomies may be more useful when performing a heart transplant. Embodiments, however, are not limited to heart or lung transplants and may be associated with any organ or structure within the thoracic cavity, such as the esophagus as one example. It should also be appreciated that the device 104 may also be used to expand an animal thoracic cavity of a non-human animal during an organ transplant and/or any other operation or procedure.

In the case of the horizontal sternotomy 110 of the thorax 108, the horizontal sternotomy 110 may be made through the sternum 112 and may separate the sternum 112 into an upper sternal portion 114 (e.g., an upper sternal segment 114, a first sternal segment 114, a first sternal portion 114, etc.) and a lower sternal portion 116 (e.g., a lower sternal segment 116, a second sternal segment 116, a second sternal portion 116, etc.). The horizontal sternotomy 110 may also separate the thoracic cage 102 into an upper thoracic cage segment or portion 118 (e.g., a first thoracic cage segment or portion 118, a first torso segment or portion 118,r an upper torso segment or portion 118, etc.) and a lower thoracic cage segment or portion 120 (e.g., a second thoracic cage segment or portion 118, a second torso segment or portion 118, a “lower torso segment or portion 118, etc.). The horizontal sternotomy 110 is typically made between two ribs 122 in the thorax 108. Between the fourth and fifth ribs 122 of the thorax 108 may be preferable for the horizontal sternotomy 110 and the placement of the device 104, but the horizontal sternotomy 110 may be made, and the device 104 may be placed, between any other pairs of ribs 122, such as between the third and fourth ribs 122.

In an embodiment, there are two or more horizontal sternotomies 110 made in the thorax 108 between two different pairs of ribs 122. In the case of two horizontal sternotomies 110, there may be a first, second, and third sternal portion as well as a first, second, and third torso portion. Additionally, in the case of two horizontal cuts, there may be two devices 104 installed in the thorax 108.

Additionally illustrated in FIG. 1 are sutures 124 that are used during closing of the thorax 108. The sutures 124 may be connected between two or more ribs 122 and may function to provide stability to the ribs 122, as the installation of the device 104 creates a gap (e.g., a sternotomy gap) between two or more ribs 122. That is, a gap formed between the ribs 122 after installation of the device 104 may be larger than the gap between the ribs 122 prior to installation of the device 104. It should be appreciated that there may be other ways of closing the gap between the ribs 122, such as additional devices 104 installed between the ribs 122, or other conventional surgical methods, such as medical staples, tapes and adhesives, skin grafts, and/or the like. In embodiments, the sutures 124 may stabilize the ribs 122 while also securing the upper thoracic cage segment 118 to the lower thoracic cage segment 120.

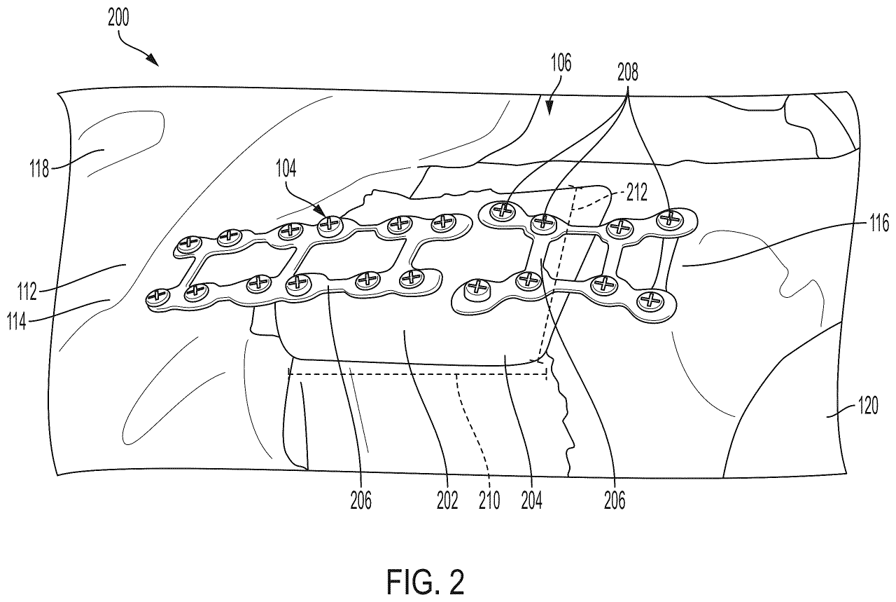

FIG. 2 is a perspective view of an embodiment of a human body 200. The human body 200 includes the installed device 104 used to expand the thoracic cavity 106 of the human body 200 during an organ transplant surgical procedure. The device 104 may include bone cement 202 that is substantially surrounded by mesh 204 (e.g., mesh envelope). The mesh 204 may function to provide the shape for the device 104 by containing the bone cement 202. The bone cement 202 and mesh 204 of the device 104 may be hand-molded or may be shaped by inserting the bone cement 202 and mesh 204 into a mold that defines a particular shape for the device 104. Additionally, the bone cement 202 and mesh 204 of the device 104 may be formed at a gentle or steep curve, but may also be substantially straight, depending on the location of the device 104. For example, the bend of the shape of the device 104 may depend on the shape, bend, and/or size of the bone that it is being attached to. In an embodiment, the appropriate length, thickness, width, shape, and/or size generally, of the device 104 may be algorithmically determined. For example, a doctor, nurse, or other operator may insert known parameters into an algorithmic model, such as the recipient's height, weight, gender, chest size, etc., as well as the dimensions of the donated organ. The algorithm may produce results as to how large the dimensions of the device 104 should be in order to sufficiently expand the thoracic cavity 106 for the installation of the organ within the recipient.

In an embodiment, the bone cement 202 is made at least partially of methyl acrylate. In some embodiments, the bone cement 202 may comprise polymethyl methacrylate (PMMA), methyl acrylate, methyl methacrylate, calcium phosphate cement, ceramic composites, or any other curable biocompatible structural materials, or any combination of the foregoing. The mesh envelope 204 may be polymeric mesh, textile mesh, metallic mesh, or composite mesh, or any combination of the foregoing. The mesh envelope 204 may further include radiopaque markers.

As is further illustrated in FIG. 2, the device 104 includes two fixation plates 206, however, it should be appreciated that the device 104 may have only one fixation plate 206 and also may have three or more fixation plates 206, depending on the application, location, and purpose of the device 104. The fixation plates 206 may be held in place by one or more fasteners 208, which in this non-limiting example include screws. The one or more fasteners 208 may attach the fixation plates 206 to the bone cement 202 and mesh 204, and the one or more fasteners 208 may attach the fixation plates 206 to the upper sternal portion 114 and/or the lower sternal portion 116 of the sternum 112. The fixation plates 206 may connect to the upper sternal portion 114 at one or more locations (e.g. points of attachment or points of anchor), and the fixation plates 206 may secure the device 104 to the upper sternal portion 114. Similarly, the fixation plates 206 may connect to the lower sternal portion 116 at one or more locations, and the fixation plates 206 may secure the device 104 to the lower sternal portion 116. In an embodiment, the device 104 requires at least three points of attachment to the upper sternal portion 114 and at least two points of attachment to the lower sternal portion 116. Accordingly, there may be a different number of points of attachment with respect to the upper sternal portion 114 and the lower sternal portion 116. Furthermore, the number of points of attachment may be based, at least in part, on a size of the device 104, a span created by the insertion of the device, and/or combinations thereof. For example, a larger device 104 (e.g., wider, thicker, longer, etc.) may use more points of attachment than a smaller device 104.

In the embodiment of the device 104 that is illustrated in FIG. 2, the device 104 has a length 210 of about 4 cm, and the device 104 has a width 212 of about 1.5 cm. In some embodiments, the length 210 of the device 104 is about 3 cm to about 5 cm long, and the width 212 of the device 104 is about 1 cm to about 2 cm. However, it should also be appreciated that the size (e.g., length 210, width 212, thickness, etc.) of the device 104 may depend on the physical size of the patient (e.g., recipient) and the size of the organ being transplanted. As such, a larger size of the device 104 may be required if particularly large lungs, for example, are being inserted in a relatively small recipient.

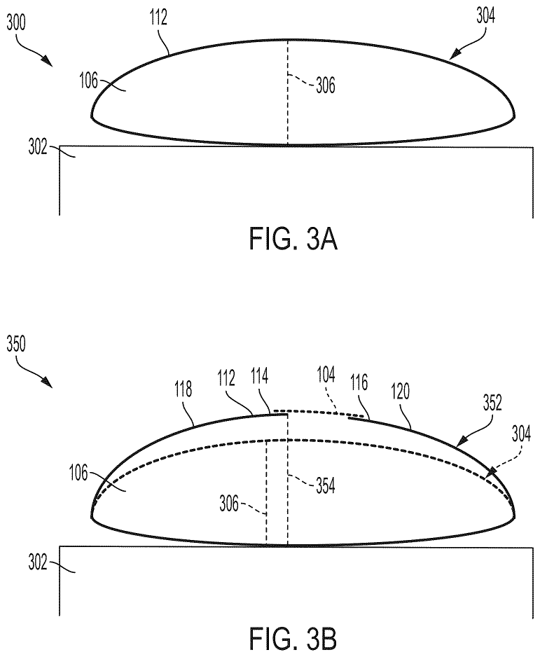

FIG. 3A is a schematic cross-sectional view of a torso 300 (e.g., the pre-surgical torso 300) of a human body. It should be appreciated that the view of FIG. 3A, showing the torso 300 and thoracic cavity 106, may not be to scale and may not be proportional to true human body dimensions, and is instead used for illustrative purposes. Additionally, FIG. 3A shows a side view of the torso 300 of a patient as if the patient were on an operating table 302.

The torso 300 of FIG. 3A defines a pre-surgical perimeter 304 (e.g., a first perimeter 304) of the torso, which generally corresponds to the surface of the upper body of a human, without including the head or arms of the human body (e.g., patient). The torso 300 further includes the thoracic cavity 106 of a pre-surgical volume (e.g., a first volume) before a surgical operation to expand the thoracic cavity 106. Although FIG. 3A is a two-dimensional illustration, one would appreciate that the pre-surgical volume of the thoracic cavity 106 will depend at least in part on a pre-surgical depth 306 (e.g., a first depth 306) of the thoracic cavity 106. It should be appreciated that the thoracic cavity 106 may not be a set volume, and instead may naturally vary due to, for example, the breathing of the human. The pre-surgical depth 306 may also not be a set value, and may vary due to the same. Furthermore, the pre-surgical volume of the thoracic cavity 106 and the pre-surgical depth 306 may vary depending on one or more of the height, gender, age, and overall lung health of the patient. For example, a six-foot, four-inch male at the age of twenty-four in good health will likely have a larger thoracic cavity 106 and depth 306 than a five-foot, two-inch female at the age of ninety-four with fibrosis in the lungs (or any other lung or heart condition).

In some embodiments, the pre-surgical volume may not be large enough to comfortably fit a donated organ within the thoracic cavity 106. It may also be the case that the pre-surgical depth 306 is similarly too small to comfortably fit the donated organ. When the donated organ is too large for the thoracic cavity 106, it can cause other health complications in the patient (e.g., recipient). Notably, the torso 300 of FIG. 3A does not contain the device 104 depicted in the post-installation embodiments of the human body 100, 200 of FIGS. 1 and 2, and the device 104 may at least partially address these issues.

FIG. 3B is a schematic cross-sectional view of a torso 350 (e.g., the post-surgical torso 350) of a human body. It should be appreciated that the view of FIG. 3B, showing the torso 350 and thoracic cavity 106, may not be to scale and may not be proportional to true human body dimensions, and is instead used for illustrative purposes. Additionally, FIG. 3B shows a side view of the torso 300 of a patient as if the patient were on the operating table 302, substantially the same operating table 302 as described in relation to FIG. 3A. Furthermore, it should be appreciated that the pre-surgical perimeter 304 and the pre-surgical depth 306 of the pre-surgical torso 300 are superimposed on the diagram of the post-surgical torso 350. A post-surgical perimeter 352 (e.g., a second perimeter 352) and a post-surgical depth 354 (e.g., a second depth) are the perimeter and depth of the post-surgical torso 350.

The torso 350 in FIG. 3B includes the thoracic cavity 106 of a post-surgical volume (e.g., a second volume) after a surgical operation to install the device 104 to expand the thoracic cavity 106. Although FIG. 3B is a two-dimensional illustration, one would appreciate that the post-surgical volume of the thoracic cavity 106 will depend at least in part on the post-surgical depth 354 of the thoracic cavity 106. It should be appreciated that the thoracic cavity 106 may not be a set volume, and instead may naturally vary due to, for example, the breathing of the human. The post-surgical depth 354 may also not be a set value, and may vary due to the similar factors.

As depicted in FIG. 3B, the torso 350 includes the sternum 112, which is split into an upper sternal portion 114 and a lower sternal portion 116 after the cut is made (as described herein) through the ribs and sternum 112 during the surgical procedure. Also as described herein, the torso 354 is also split into an upper thoracic cage portion 118 and a lower thoracic cage portion 120 after the same cut is made. The device 104 attaches to the upper sternal portion 114 and to the lower sternal portion 116, thereby connecting the two sternal portions 112, 114.

As discussed herein, the device 104 may function by increasing the volume of the thoracic cavity 106 after a surgical procedure. As can be seen in FIG. 3B, the superimposed pre-surgical depth 306 of the torso cavity 104 is smaller than the post-surgical depth 354, thereby signifying an increase in the volume of the thoracic cavity 106. As can be also seen in the view of FIG. 3B, the superimposed pre-surgical perimeter 304 is smaller than the post-surgical perimeter 352. It may be the case that the length of the device 104 plus the length of the pre-surgical perimeter 304 equals the post-surgical perimeter 352. However, in some embodiments, the device 104 at least partially overlaps the upper sternal portion 114 and/or the lower sternal portion 116. In these embodiments, the length of the device 104 plus the length of the pre-surgical perimeter 304 would be larger than the post-surgical perimeter 352. In some embodiments, depending on the geometrical shape of the pre-surgical perimeter 304 and desired post-surgical perimeter 352, the required length of the device 104 installed during the surgical procedure may be calculated via algorithmic models. Similarly, it may be possible to calculate the relative increase in depth of the thoracic cavity 106 that would be realized based on the size of the device 104 installed.

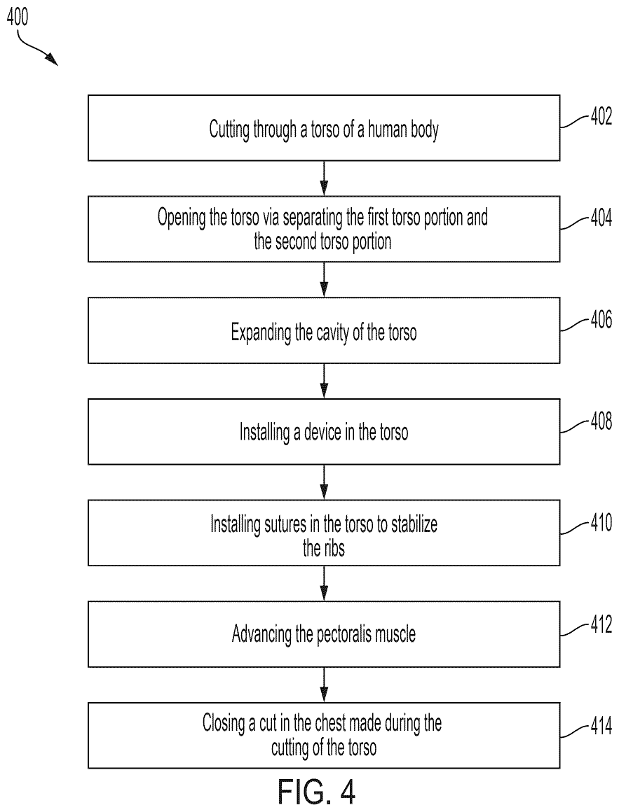

FIG. 4 is a flow chart of a surgical method 400 for expanding a thoracic cavity of a thorax of a human body. It should be appreciated that steps for the method, and any other method discussed herein, may be performed in any order, or in parallel, unless otherwise specifically stated. Moreover, the method, and any other method discussed herein, may include more or fewer steps. In this example, cutting through a torso of the human body 402 may be performed as part of the method 400. The torso may include a thoracic cavity, and the cutting of the torso creates a first torso portion, a second torso portion, a first sternal portion, and a second sternal portion. Further, the surgical method 400 may include opening the torso via separating the first torso portion and the second torso portion 404. The surgical method 400 may also include expanding the cavity of the torso 406. In at least one embodiment, expanding the cavity of the torso may include increasing a distance between the first torso portion and the second torso portion and/or increasing a distance between the first sternal portion and the second sternal portion. Additionally, the surgical method 400 may include installing a device in the torso 408, the device being positioned between the first torso portion and the second torso portion and holding the first torso portion and the second torso portion apart from each other. The device that is installed in the surgical method 400 may include bone cement positioned between the first sternal portion and the second sternal portion, one or more fixation plates, and one or more fasteners. The one or more fixation plates of the device installed in the surgical method 400 may be are attachable to the first sternal portion, the second sternal portion, and the bone cement via the one or more fasteners.

In some embodiments, the surgical method 400 additionally includes installing sutures in the torso to stabilize the ribs 410. In such embodiments, the sutures may be positioned between the first torso portion and the second torso portion. In an embodiment, the surgical method 400 also includes advancing the pectoralis muscle 412, which may be necessitated at least in part due to the expansion of the thoracic cavity. In another embodiment, the surgical method 400 further includes closing a cut in the thorax made during the cutting of the torso 414.

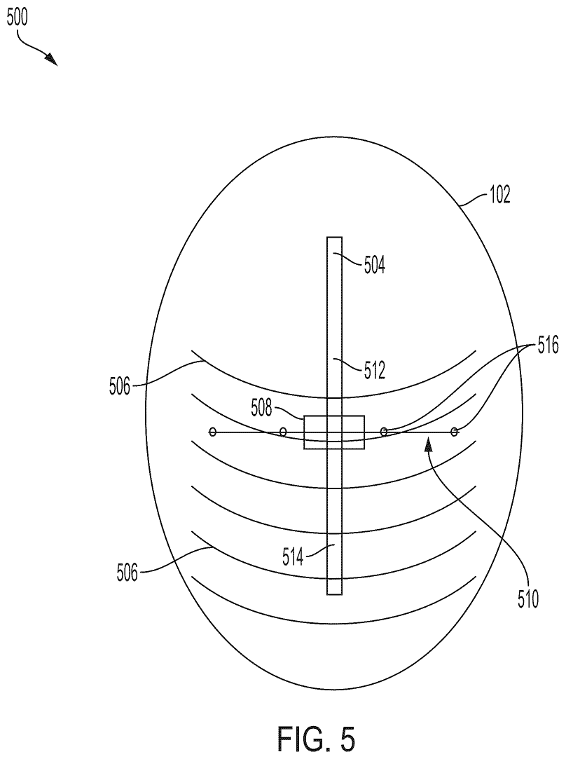

FIG. 5 is a schematic view of a post-surgical system 500 of a torso 502 having a sternum 504 and multiple ribs 506, the system 500 including a thoracic expansion device 508 (e.g., expansion device, device 508, etc.) positioned across a sternotomy gap 510. The sternotomy gap 510 may separate the sternum 504 into an upper sternal segment 512 and a lower sternal segment 514. The system 500 may further include one or more rib-stabilizing sutures 516 that may be positioned on one or both sides of the device 508 in order to stabilize the ribs 506. Although four stabilizing sutures 516 are depicted in FIG. 5, there may be any reasonable number of rib-stabilizing sutures 516, which may depend on various factors such as placement of the cut in the torso, or size of the thoracic expansion device or sternotomy gap, as just a few examples. The sutures 516 may also secure an upper thoracic cage segment to a lower thoracic cage segment, as described above with respect to FIG. 1.

As used herein, a thoracic cage segment may include any bony and/or cartilaginous portion of the thoracic cage that can be surgically separated to create a gap, including one or more sternal segments, rib segments, costal cartilage, or combinations thereof. Additionally, a sternotomy gap may include a gap created by a transverse sternotomy, a median sternotomy, a clamshell incision, or any surgical cut that separates thoracic cage segments to permit access and/or expansion.

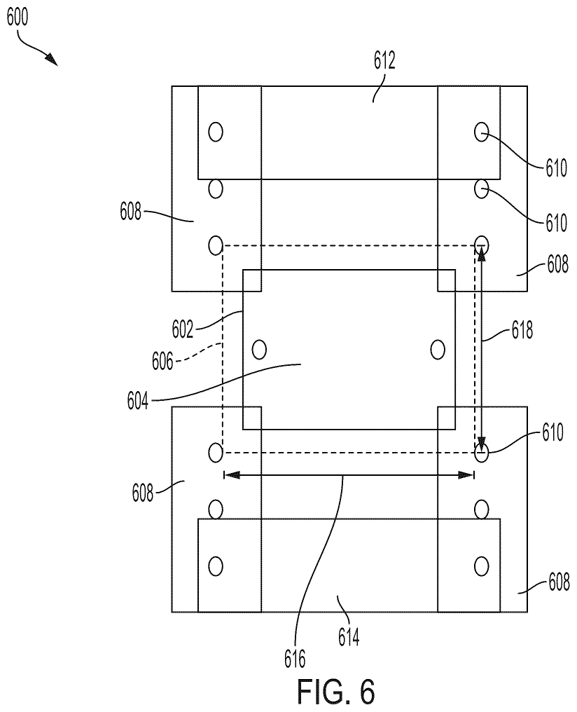

FIG. 6 is a schematic view of a system 600 capable of expanding the thoracic cavity of a body during a surgical operation. The system 600 includes a thoracic expansion device 602 (e.g., a device), which, as described herein, may be made of bone cement 604 and may be enveloped in mesh 606 (e.g., a mesh envelope). The system 600 may further include one or more fixation plates 608 (e.g., plates), which may allow the device 602 to be affixed to one or more sternal portions and/or one or more ribs (not depicted in FIG. 6) during a thoracic cavity expansion surgical operation. In the embodiment depicted in FIG. 6, there are two plates 608 positioned above the device 602 (e.g., on the upper side of the device 602) and there are two plates 608 positioned below the device 602 (e.g., on the lower side of the device 602). However, it should be appreciated that there may be any reasonable number of fixation plates employed to securely affix the device 602 to a thorax during the surgical operation. The plates 608 may be affixed using one or more fasteners 610, which may be screws, clamps, or any other fastening apparatus. In the embodiment illustrated in FIG. 6, there are three fasteners 610 per fixation plate 608, however, it should be appreciated that the example configuration is provided for illustrative purposes and there may be any reasonable number of fasteners 610 used in the system 600.

The system 600 may also include an upper section 612 which may be positioned across the upper end of the two upper fixation plates 608 and a lower section 614 which may be positioned across the lower end of the two lower fixation plates 608. The upper and lower sections 612, 614 may function to stabilize the device 602 on an upper sternal segment and a lower sternal segment, respectively.

Furthermore, the thoracic expansion device 602, including the bone cement 604 and mesh envelope 606 may be have a certain width 616 and length 618, as described herein. The dimensions, such as the width 616 and height 618, may be selected based on a target distraction distance and/or a contact area desired for load distribution. The device 602 comprising the bone cement 604 and mesh envelope 606 may be molded intraoperatively (e.g., during an organ transplant surgical procedure itself), on an ad hoc basis, in order to fit the particular recipient of the organ transplant. For example, if the recipient is smaller than average and is receiving a relatively larger organ (e.g., lungs) during the transplant, the thoracic cage of the recipient may need to be expanded larger. As such, the device 602, with the bone cement 604 and the mesh envelope 606, may be molded intraoperatively to be larger (e.g., larger width 616 and/or larger length 618) in order to span the larger sternotomy gap created to fix the larger organ into the thoracic cavity. Additionally, the device 602, with the bone cement 604 and the mesh envelope 606, may be molded to match the curvature of the chest wall or the thoracic cage. However, in various embodiments, the device 602, and/or portions thereof, may be fabricated in predetermined sizes, such as a small, medium, or large size, among other options, and may be provided as part of a kit or set for use during surgery. Providing preset sizes for components may enable simplified manufacturing and may also facilitate the use of surgical grade materials that may be sterilized and shipped. Furthermore, in at least one embodiment, different segments may be provided that can be joined together to enable adjustments during surgical procedures, such as including the bone cement 604 and mesh envelope 606 with a preset width 616 and/or length 618 that may be coupled together (e.g., along the length 618) to accommodate a variety of different dimensions.

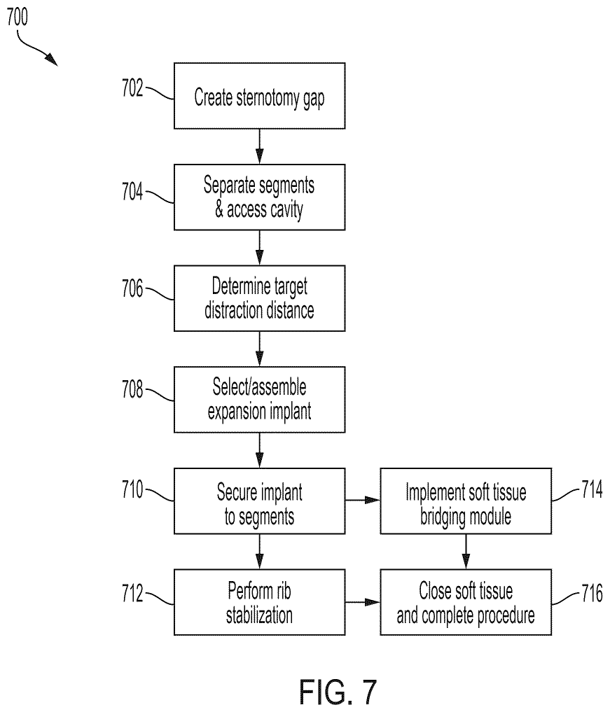

FIG. 7 is a flow chart of a method 700 for thoracic cavity expansion during a surgical operation. In this example, creating a sternotomy gap 702 may be performed as part of the method 700, which may include cutting into the thoracic cage in a predetermined location. Further, the method 700 may include separating two segments of the thoracic cage (e.g., an upper segment and a lower segment, as described herein) created by the sternotomy gap and accessing the thoracic cavity 704.

The method 700 may also include determining a target distraction distance 706. The distraction distance (e.g., distraction height, distraction length, etc.) may refer to a maintained separation distance between opposing cut surfaces of thoracic cage segments. The distraction distance may be fixed, adjustable, continuous, or quantized (e.g., in standardized module increments). Furthermore, the method 700 may include selecting and assembling the expansion implant (e.g., the thoracic expansion device) 708, which may be based, at least partially, on the target distraction distance. For example, if a smaller distraction distance is required due to a smaller sternotomy gap, the expansion implant/device selected, formed, assembled, etc. may be relatively smaller.

In some embodiments for determining target distraction distance, a predictive sizing workflow receives recipient data (e.g., height, weight, sex, imaging-derived thoracic cavity geometry/volume) and donor data (e.g., donor lung geometry/volume). A volumetric mismatch value is computed and mapped to an outputted target distraction distance. The output can specify a monolithic spacer geometry, a modular module count, and/or a pre-defined implant category. The volumetric mismatch may include any metric representing a difference between recipient thoracic capacity and donor organ size. In some embodiments, volumetric mismatch is computed as donor lung volume minus recipient thoracic cavity volume. In other embodiments, volumetric mismatch is derived from imaging, predicted total lung capacity, or other geometry-based surrogates.

In some embodiments, the mapping of the target distraction distance is represented by a function D=f(Vₘ, G), where Dis distraction distance, Vₘ is volumetric mismatch, and G represents one or more geometric parameters (e.g., anterior-posterior depth, perimeter, cross-sectional area, etc.). In some embodiments, the system outputs a recommended module count N=round(D/H), where H is a standardized module height, and the module count is rounded to the nearest whole number. In some embodiments, the workflow is performed by a computing system and/or by manual templates and sterile sizing gauges. In addition, manual sizing can be performed intraoperatively by inserting a sterile gauge into the gap to verify the distraction distance prior to implant insertion.

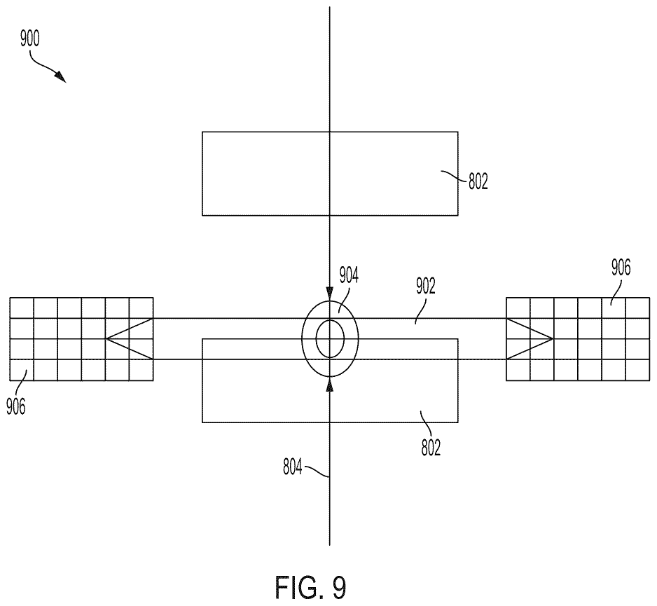

Additionally, the method 700 may include securing the expansion implant or the thoracic expansion device to the thoracic cage segments 710. As mentioned herein, this may be accomplished using fixation plates, fasteners, or other methods. Optionally, in some embodiments, the method 700 may include performing rib stabilization 712 during the surgical procedure. Furthermore, in some embodiments, the method 700 may include implementing a soft tissue bridging module 714. As discussed further with respect to FIG. 9, the soft tissue bridging module may be any scaffold, skirt, mesh, sheet, flap interface, or porous structure that facilitates closure and/or coverage of an expansion construct by muscle, fascia, dermis, or other tissues. The method 700 may also include closing the soft tissue and completing the surgical procedure 716.

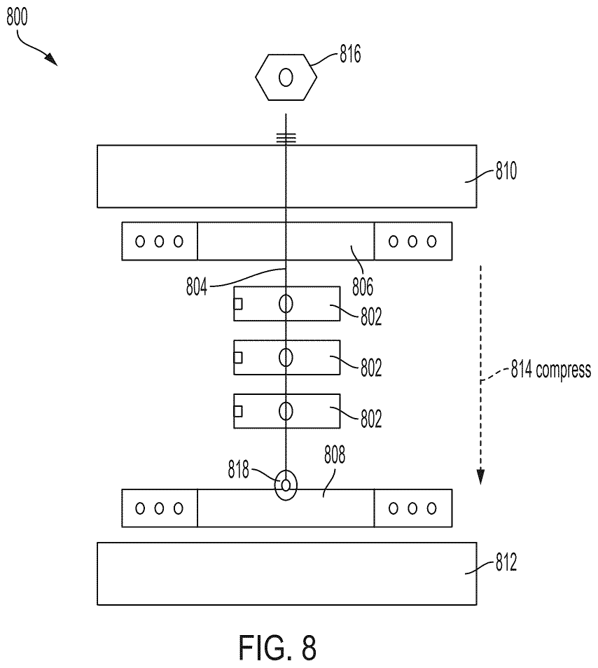

FIG. 8 is an exploded schematic view of a modular thoracic expansion system 800 that includes one or more stackable spacer modules 802 (e.g., spacer modules, modules, etc.) and a central retention rod 804 (e.g., a retention rod, a rod, etc.). The one or more stackable spacer modules 802 may be centered along the central retention rod 804 and positioned between a cap platform 806 and a base platform 808. It should be appreciated that although there are three stackable spacer modules 802 depicted in FIG. 8, there may be any reasonable number of modules 802. A greater number of modules 802 may be required when the system 800 is required to span a larger sternotomy gap, and vice versa.

The cap platform 806 may be secured to a first sternal segment 810 of a recipient's sternum, and the base platform 808 may be secured to a second sternal segment 812 of a recipient's sternum. The central retention rod 804 may extend between the platforms 806, 808. When making up the modular expansion system 800, the components of the system 800 (such as the modules 802, base platform 808, and cap platform 806) may be compressed together 814 along the retention rod 804. Embodiments may include washers or packing material between the stackable spacer modules 802, between the stackable spacer modules 802 and the cap platform 806, and/or between the stackable spacer modules 802 and the base platform 808. A locking nut 816 (or other locking mechanism) may apply a compressive force to the system 800, forming a rigid, load-bearing column that may maintain a selected distraction height based on the number or size of modules 802 selected for the system 800.

In embodiments, each spacer module 802 defines a standardized height increment (e.g., about 10 mm). In some embodiments, some modules 802 may have a fraction of the standardized height increment, for example, a half-size module. In some embodiments, standardized modular increments permit rapid intraoperative adjustment and enable pre-defined category bins (e.g., less than about 3 cm, about 3-6 cm, and greater than about 6 cm), while still allowing fine tuning by selecting module counts and/or mixed module thicknesses. In addition, the modules 802 may be keyed or textured to resist rotation and shear. In some embodiments, the modules 802 may interlock, snap together, or otherwise connect or secure to each other.

In some embodiments, the base platform 808 may include a polyaxial head 818 to permit angulation of the rod 804 to accommodate non-planar sternal geometries. For example, if the cap platform 806 and base platform 808 secured to their respective sternal portions are not parallel or aligned, the polyaxial head 818 may allow the rod 804 (and thus the expansion construct) to be angled appropriately so that the system fits securely and distributes force evenly.

In some embodiments, the modular thoracic expansion system is configured for temporary use or rapid removal. For example, an inflatable bladder may be integrated and positioned in the sternotomy gap and adjusted through a port accessible from the exterior of the thoracic cage. In some embodiments, the fixation of various elements in the thoracic expansion device may be quick-release (e.g., removable clips or biodegradable sutures), allowing collapse and withdrawal of the thoracic expansion device to facilitate rapid closure or re-exploration.

FIG. 9 is a schematic view of a soft tissue bridging module 900 captured between two adjacent spacer modules 802. The soft tissue bridging module 900 may include a flexible sheet body 902 with a reinforced grommet 904 configured to receive the central retention rod 804. The flexible sheet body 902 with the reinforced grommet 904 may be positioned between adjacent spacer modules 802 and retained by compressive force, for example, from the compression by the locking nut, as explained in regards to FIG. 8. The soft tissue bridging module may also include one or more lateral porous lattice extensions 906 that may be included in the module 900 to provide suture purchase to attach pectoralis, fascia, dermis, or other tissues. In other words, the one or more porous lattice extensions 906 may be designed to allow sutures to be passed through them and to hold those sutures securely.

In some embodiments, the soft tissue bridging module 900 may comprise a bioabsorbable polymer mesh, a permanent synthetic mesh, a biologic scaffold (including acellular dermal matrix), or combinations thereof. In some embodiments, the biologic scaffold may include antibiotic elution and/or surface treatments that reduce bacterial adherence, which thereby may reduce the risk of infection.

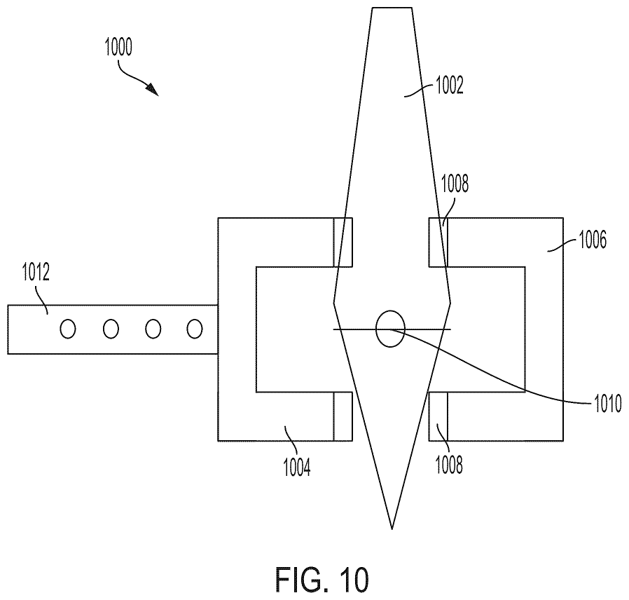

FIG. 10 is a schematic view of a distal anchoring clamp 1000 (e.g., a clamp) that may be configured to engage a xiphoid process or costal margin region 1002 of a patient undergoing a surgical procedure, which may be employed when the patient has poor bone density such that fixation of a thoracic expansion device solely to sternal portions is not sufficient to properly anchor the device to create the expanded thoracic cavity. In other embodiments, the distal anchoring clamp 1000 may be implemented to achieve a screwless fixation to at least a portion of the xiphoid process, costal margin region, or sternum, opting for a jaw configuration instead without penetrating some of the xiphoid process, costal margin region, or sternum of the patient's torso.

The clamp 1000 may include one or more jaws 1004, 1006, which may be characterized by a first jaw 1004 and a second jaw 1006, but it should be appreciated that there may be any reasonable number of jaws incorporated into the clamp 1000. In the embodiment illustrated in FIG. 10, the first jaw 1004 is substantially opposed to the second jaw 1006 and the jaws 1004, 1006 are gripping opposite sides of the xiphoid process or costal margin region 1002. Grip between the jaws 1004, 1006 and the xiphoid process or costal margin region 1002 may be accomplished via one or more friction or textured pads 1008. In addition to a clamp 1000 with jaws 1004, 1006, other screwless fixation embodiments may include cerclage cables, straps, sternal wires, adhesive interfaces, or hybrid constructs that distribute load across a larger bone surface area to reduce pull-out risk in osteopenic bone (e.g., bone that is less dense and more fragile).

Further depicted in FIG. 10, the distal anchoring clamp 1000 may include a clamp actuator 1010 that may function to bring the first jaw 1004 toward the second jaw 1006 and apply a clamping force upon the xiphoid process or costal margin region 1002. There may also be more than one actuator 1010, which may function to move the first jaw 1004 and the second jaw 1006 separately. In embodiments, the actuator 1010 may be a manual screw actuator, a ratchet mechanism, a spring-loaded actuator, or a lever/cam actuator, as these examples may be reliable, easy to control, and/or suitable for sterile environments. The clamp 1000 may further include an interface arm 1012 that may be designed to secure the clamp 1000 to the thoracic expansion device. As such, the clamp 1000 assembly may be an add-on feature of the thoracic expansion device that could be used in particular use cases. The interface arm 1012 may attach to a fixation plate or one or more modules of the thoracic expansion device to incorporate the clamp 1000 into a the larger surgical system.

### NON-LIMITING EXAMPLE EMBODIMENTS

One or more embodiments of the present disclosure may further be described in view of the follow clauses associated with non-limiting example embodiments:
1. A thoracic cavity expansion implant comprising:
a spacer assembly configured to be positioned between a first thoracic cage segment and a second thoracic cage segment that are separated during a surgical operation, the spacer assembly defining a distraction distance that increases a thoracic cavity volume; and
a fixation assembly configured to secure the spacer assembly to the first thoracic cage segment and the second thoracic cage segment to maintain the distraction distance.
2. The implant of clause 1, wherein the first thoracic cage segment and the second thoracic cage segment comprise a first sternal segment and a second sternal segment, respectively.
3. The implant of clause 1, wherein the fixation assembly comprises one or more fixation plates configured for attachment to an anterior surface of the thoracic cage segments.
4. The implant of clause 3, wherein the fixation assembly comprises bone screws.
5. The implant of clause 3, wherein the fixation assembly comprises a screwless fixation arrangement comprising at least one of a clamp, a strap, a cable, a wire, or an adhesive interface.
6. The implant of clause 1, wherein the spacer assembly comprises a monolithic spacer body.
7. The implant of clause 6, wherein the monolithic spacer body comprises a curable material contained by a mesh envelope.
8. The implant of clause 7, wherein the curable material comprises polymethyl methacrylate (PMMA), methyl acrylate, methyl methacrylate, calcium phosphate cement, or ceramic composite cement.
9. The implant of clause 1, wherein the spacer assembly is formed to have a curved profile corresponding to chest wall curvature at an implant site.
10. The implant of clause 1, wherein the implant further comprises rib-stabilizing sutures configured to stabilize an intercostal gap enlarged by the distraction distance.
11. The implant of clause 1, wherein the spacer assembly comprises an antimicrobial surface treatment or an antibiotic-eluting material configured to reduce infection risk.
12. The implant of clause 1, wherein the fixation assembly provides at least three fixation points on a first side of a sternotomy and at least two fixation points on a second side of the sternotomy.
13. The implant of clause 1, further comprising a quick-release interface configured to permit removal or exchange of the spacer assembly without removing bone-attached hardware.
14. The implant of clause 1, wherein the spacer assembly includes one or more radiopaque markers.
15. The implant of clause 1, wherein the distraction distance is selected to be less than about 3 cm, from about 3 cm to about 6 cm, or greater than about 6 cm.
16. The implant of clause 1, wherein the spacer assembly is configured to maintain the distraction distance during closure of soft tissue over the implant.
17. A modular thoracic cavity expansion system comprising:
a base platform configured for fixation to a first sternal segment;
a cap platform configured for fixation to a second sternal segment;
a plurality of stackable spacer modules each defining a standardized module height and each comprising a central pass-through aperture; and
a central retention rod configured to extend between the base platform and the cap platform through the respective central pass-through apertures of the plurality of stackable spacer modules;
wherein the central retention rod includes a locking mechanism configured to compress the plurality of stackable spacer modules into a rigid load-bearing column that maintains a selected distraction distance between the first sternal segment and the second sternal segment.
18. The system of clause 17, wherein the standardized module height is about 10 mm.
19. The system of clause 17, wherein at least one of the base platform or the cap platform comprises lateral flanges having fastener holes configured to receive bone screws.
20. The system of clause 17, wherein the locking mechanism comprises a threaded shaft portion of the central retention rod and a locking nut configured to thread onto the threaded shaft portion.
21. The system of clause 17, wherein the base platform comprises a polyaxial head configured to permit angulation of the central retention rod relative to the base platform.
22. The system of clause 17, wherein the spacer modules comprise keyed interlocking features on opposing faces configured to resist rotation of the spacer modules relative to one another about the central retention rod.
23. The system of clause 17, wherein the spacer modules comprise surface textures configured to increase frictional resistance to shear displacement.
24. The system of clause 17, further comprising a soft-tissue bridging module having a reinforced central aperture configured to receive the central retention rod and lateral porous extensions configured for suturing soft tissue.
25. The system of clause 24, wherein the soft-tissue bridging module is configured to be retained by compressive force between adjacent spacer modules.
26. The system of clause 17, further comprising a distal anchoring clamp coupled to the base platform and configured to grip a xiphoid process or costal margin without a penetrating fastener.
27. The system of clause 26, wherein the distal anchoring clamp comprises opposed jaws and an actuator configured to generate clamping force.
28. The system of clause 17, wherein at least one of the spacer modules comprises a radiopaque marker indicating the standardized module height.
29. The system of clause 17, wherein the spacer modules are provided as a sterile kit including a plurality of different module heights.
30. The system of clause 17, wherein the system is configured for use with a transverse sternotomy or a clamshell incision.
31. The system of clause 17, wherein the cap platform is configured to couple to the central retention rod via a threaded engagement, a bayonet lock, a cam lock, or a ratcheting collet.
32. The system of clause 17, wherein the rigid load-bearing column defines a distraction distance selected from less than about 3 cm, about 3 cm to about 6 cm, and greater than about 6 cm.
33. The system of clause 17, wherein the system further comprises an antimicrobial coating on at least one of the spacer modules, the base platform, or the cap platform.
34. The system of clause 17, wherein at least one of the base platform or the cap platform is configured for screwless fixation to a sternal segment.
35. The system of clause 17, wherein the spacer modules are configured to be assembled intraoperatively to achieve a custom distraction distance.
36. A method for selecting a patient-specific thoracic expansion construct, comprising: receiving recipient data comprising at least one of a recipient chest cavity volume or recipient thoracic geometry;
receiving donor data comprising at least one of a donor lung volume or donor lung geometry;
calculating a volumetric mismatch value based on the recipient data and the donor data;
determining, using the volumetric mismatch value, a target distraction distance; and
selecting, based on the target distraction distance, at least one of (i) a modular spacer module count, (ii) a monolithic spacer geometry, or (iii) a selection among predefined implant categories.
37. The method of clause 36, wherein calculating the volumetric mismatch value comprises subtracting the recipient chest cavity volume from the donor lung volume.
38. The method of clause 36, wherein selecting among predefined implant categories comprises selecting a small category less than about 3 cm, a medium category from about 3 cm to about 6 cm, or a large category greater than about 6 cm.
39. The method of clause 36, wherein determining the target distraction distance comprises applying a predictive expansion algorithm.
40. The method of clause 39, wherein the predictive expansion algorithm computes the target distraction distance as a function of the volumetric mismatch value and one or more thoracic geometry parameters.
41. The method of clause 36, wherein receiving recipient data comprises receiving imaging-derived geometry from CT or MRI.
42. The method of clause 36, wherein receiving donor data comprises receiving imaging-derived donor lung geometry.
43. The method of clause 36, wherein selecting comprises outputting a module count for spacer modules having a standardized height of about 10 mm.
44. The method of clause 36, further comprising verifying the target distraction distance intraoperatively using a sterile gauge temporarily inserted into a sternotomy gap.
45. The method of clause 36, further comprising outputting a recommended fixation modality based on bone density indicators.
46. The method of clause 45, wherein the fixation modality is selected from screws, cerclage cables, straps, clamps, or adhesive interfaces.
47. The method of clause 36, further comprising outputting a recommended soft-tissue closure strategy including use of a soft-tissue bridging module.
48. The method of clause 36, further comprising outputting a curvature specification for the expansion construct.
49. The method of clause 36, wherein selecting includes selecting a distal anchoring clamp when distal sternal bone quality is below a threshold.
50. The method of clause 36, wherein selecting includes selecting a temporary or removable expander when a bleeding-risk indicator exceeds a threshold.
51. The method of clause 36, further comprising generating a kit pick-list identifying sterile components to open for the procedure.
52. The method of clause 36, wherein the recipient data includes at least one of height, weight, sex, chest size, or a fibrosis/scarring indicator.
53. The method of clause 36, wherein the donor data includes at least one of donor height, donor sex, donor predicted total lung capacity, or a donor lung volume proxy.
54. The method of clause 36, further comprising storing the target distraction distance and the selected construct in a patient record.
55. The method of clause 36, wherein the method is performed at least in part by a computing device.
56. A method for expanding a thoracic cavity during a surgical operation, comprising:
creating a sternotomy gap between a first sternal segment and a second sternal segment;
separating the first sternal segment and the second sternal segment to increase a thoracic cavity volume;
positioning a thoracic expansion implant in the sternotomy gap to establish a distraction distance;
securing the thoracic expansion implant to the first sternal segment and the second sternal segment to maintain the distraction distance; and
closing soft tissue over the thoracic expansion implant.
57. The method of clause 56, wherein creating the sternotomy gap comprises performing a transverse sternotomy.
58. The method of clause 56, wherein creating the sternotomy gap comprises performing a clamshell incision.
59. The method of clause 56, wherein positioning the thoracic expansion implant comprises installing a monolithic cement spacer contained within mesh.
60. The method of clause 56, wherein positioning the thoracic expansion implant comprises assembling a modular column from stackable spacer modules on a central retention rod.
61. The method of clause 56, further comprising placing rib-stabilizing sutures between ribs adjacent the sternotomy gap.
62. The method of clause 56, further comprising implanting donor lungs after securing the thoracic expansion implant.
63. The method of clause 56, further comprising implanting a donor heart after securing the thoracic expansion implant.
64. The method of clause 56, further comprising installing a soft-tissue bridging module retained by the thoracic expansion implant and suturing soft tissue to porous lattice extensions of the soft-tissue bridging module.
65. The method of clause 56, further comprising engaging a distal anchoring clamp that grips a xiphoid process or costal margin without screws.
66. The method of clause 56, wherein securing comprises selecting a screw fixation, a screwless fixation, or a hybrid fixation based on bone quality.
67. The method of clause 56, further comprising selecting the distraction distance based on recipient thoracic capacity and donor lung size prior to positioning the thoracic expansion implant.
68. The method of clause 56, further comprising selecting the distraction distance intraoperatively using a sterile sizing gauge.
69. The method of clause 56, wherein the thoracic expansion implant comprises an antimicrobial surface treatment or an antibiotic-eluting material.
70. The method of clause 56, wherein the distraction distance is less than about 3 cm, from about 3 cm to about 6 cm, or greater than about 6 cm.
71. The method of clause 56, further comprising using a temporary expander in the sternotomy gap and removing the temporary expander prior to final closure.
72. The method of clause 56, further comprising removing or exchanging the thoracic expansion implant via a quick-release interface during a re-exploration procedure.
73. The method of clause 56, further comprising applying a negative-pressure dressing over a closed incision following closure.
74. The method of clause 56, further comprising placing a biologic scaffold over the thoracic expansion implant prior to closure.
75. The method of clause 56, wherein securing comprises distributing load across osteopenic bone using an enlarged plate footprint or an adhesive-augmented interface.
76. A soft-tissue bridging module for use with a thoracic expansion construct, comprising:
a flexible sheet body having a medial portion and a lateral extension;
a reinforced central aperture in the medial portion configured to receive a retention rod or a fastener of the thoracic expansion construct;
wherein the flexible sheet body is configured to be mechanically retained by the thoracic expansion construct; and
wherein the lateral extension comprises a porous lattice configured to receive sutures for attaching soft tissue.
77. The module of clause 76, wherein the reinforced central aperture comprises a grommet formed from titanium or polyether ether ketone (PEEK).
78. The module of clause 76, wherein the porous lattice comprises a bioabsorbable polymer mesh configured to promote tissue ingrowth.
79. The module of clause 76, wherein the porous lattice comprises a permanent synthetic mesh configured to provide long-term reinforcement.
80. The module of clause 76, wherein the flexible sheet body comprises an acellular dermal matrix or a composite of biologic and synthetic layers.
81. The module of clause 76, wherein the flexible sheet body includes antibiotic elution.
82. The module of clause 76, wherein the flexible sheet body includes a low-biofilm surface texture.
83. The module of clause 76, wherein the lateral extension comprises suture tabs positioned to align with pectoralis, fascial, or dermal edges.
84. The module of clause 76, wherein the module is configured to be sandwiched between adjacent spacer modules of a modular column.
85. The module of clause 76, wherein the module is configured to be secured to a fixation plate of a cement spacer construct.
86. The module of clause 76, wherein the module further comprises radiopaque markers.
87. The module of clause 76, wherein the module comprises an integrated barrier layer configured to reduce adhesions.
88. The module of clause 76, wherein the module is configured to be trimmed intraoperatively while preserving suture lattice integrity.
89. The module of clause 76, wherein the porous lattice comprises a lattice pattern selected from a grid, a diamond mesh, or a radial spoke pattern.
90. The module of clause 76, wherein the module is provided sterile-packaged and labeled for a predefined distraction category.
91. A distal anchoring clamp for a thoracic expansion construct, comprising:
a first jaw and a second jaw configured to engage a xiphoid process or costal margin region;
an actuator configured to generate clamping force between the first jaw and the second jaw without a penetrating bone fastener; and
a construct interface configured to couple the clamp to a thoracic expansion implant to resist migration of the implant.
92. The clamp of clause 91, wherein the first jaw and the second jaw include friction pads or textured surfaces configured to increase resistance to slippage.
93. The clamp of clause 91, wherein the actuator comprises a screw mechanism.
94. The clamp of clause 91, wherein the actuator comprises a cam-lock mechanism.
95. The clamp of clause 91, wherein the construct interface comprises a plate arm having fastener holes for coupling to a base platform or fixation plate.
96. The clamp of clause 91, wherein the clamp includes a load-distributing jaw footprint configured for osteopenic anatomy.
97. The clamp of clause 91, wherein the clamp is configured to engage costal cartilage.
98. The clamp of clause 91, wherein the clamp is configured to engage tendinous or fascial tissue adjacent the xiphoid process.
99. The clamp of clause 91, wherein the clamp includes an adjustable jaw spacing to accommodate variable anatomy.
100. The clamp of clause 91, wherein the clamp comprises radiopaque markers.
101. The clamp of clause 91, wherein the clamp comprises an antimicrobial coating.
102. The clamp of clause 91, wherein the clamp is configured for tool-less release after implantation.
103. The clamp of clause 91, wherein the clamp is configured to cooperate with cerclage cables or straps.
104. The clamp of clause 91, wherein the clamp is provided as part of a sterile kit with spacer modules.
105. The clamp of clause 91, wherein the clamp includes a breakaway feature configured to limit clamping force and reduce tissue injury.
106. A temporary thoracic expansion system comprising:
an expandable element configured to be positioned within a sternotomy gap to maintain a temporary distraction distance;
a fluid port configured to permit adjustment of the expandable element after placement; and
a fixation arrangement configured for rapid release;
wherein the expandable element is configured to be collapsed and withdrawn to permit rapid chest closure or re-exploration.
107. The system of clause 106, wherein the expandable element comprises an inflatable bladder.
108. The system of clause 106, wherein the fluid port is configured to remain accessible from an exterior of a patient.
109. The system of clause 106, wherein the fixation arrangement comprises removable clips.
110. The system of clause 106, wherein the fixation arrangement comprises biodegradable sutures.
111. The system of clause 106, wherein the expandable element comprises a pressure-limiting feature configured to limit distraction force.
112. The system of clause 106, wherein the expandable element comprises radiopaque markers.
113. The system of clause 106, wherein the expandable element comprises an antimicrobial surface treatment.
114. The system of clause 106, wherein the system is configured to be replaced with a permanent spacer construct after a bleeding-risk period.
115. The system of clause 106, further comprising a quick-release coupler configured to connect the temporary system to a base platform or fixation plate.
116. The system of clause 106, wherein the expandable element is configured to be removed through a limited incision.
117. The system of clause 106, wherein the expandable element comprises an internal baffle configured to reduce migration.
118. The system of clause 106, wherein the expandable element comprises a shape selected to match a sternal curvature.
119. The system of clause 106, wherein the temporary distraction distance is adjustable between about 1 cm and about 8 cm.
120. The system of clause 106, wherein the fluid port is configured for connection to a sterile syringe or pump.
121. A surgical kit for thoracic cavity expansion, comprising:
a plurality of sterile-packaged thoracic expansion constructs corresponding to discrete distraction distance ranges; and
at least one sterile sizing instrument configured to be temporarily inserted into a sternotomy gap to verify a distraction distance prior to selecting one of the thoracic expansion constructs.
122. The kit of clause 121, wherein the discrete distraction distance ranges include a first range less than about 3 cm, a second range from about 3 cm to about 6 cm, and a third range greater than about 6 cm.
123. The kit of clause 121, wherein at least one of the thoracic expansion constructs comprises a modular system including spacer modules and a central retention rod.
124. The kit of clause 121, wherein at least one of the thoracic expansion constructs comprises a cement spacer contained within mesh and associated fixation plates.
125. The kit of clause 121, wherein the sizing instrument comprises a stepped gauge corresponding to standardized module increments.
126. The kit of clause 121, further comprising a plurality of soft-tissue bridging modules configured for use with the thoracic expansion constructs.
127. The kit of clause 121, further comprising a distal anchoring clamp configured to grip a xiphoid process or costal margin.
128. The kit of clause 121, further comprising a selection guide mapping distraction distance to module count.
129. The kit of clause 121, wherein each sterile package includes machine-readable labeling encoding distraction range and curvature profile.
130. The kit of clause 121, wherein the kit further comprises cerclage cables or straps for screwless fixation.
131. The kit of clause 121, wherein the kit further comprises an antimicrobial irrigation or coating application component.
132. The kit of clause 121, wherein the kit includes a temporary inflatable expander.
133. The kit of clause 121, wherein the kit includes a quick-release interface component for spacer exchange.
134. The kit of clause 121, wherein the kit includes spacer modules of multiple thicknesses to provide fine adjustment.
135. The kit of clause 121, wherein the kit includes a sterile template for matching curvature to a patient anatomy.
136. A non-transitory computer-readable medium storing instructions that, when executed by one or more processors, cause a system to:
receive recipient thoracic geometry data and recipient thoracic volume data;
receive donor lung geometry data and donor lung volume data;
compute a volumetric mismatch value;
compute a target distraction distance based on the volumetric mismatch value; and
output a recommended thoracic expansion construct including at least one of a module count, a predefined size category, a curvature specification, or a fixation modality selection.
137. The non-transitory computer-readable medium of clause 136, wherein the recipient thoracic geometry data comprises CT-derived geometry.
138. The non-transitory computer-readable medium of clause 136, wherein the system outputs a module count for spacer modules of about 10 mm height.
139. The non-transitory computer-readable medium of clause 136, wherein the predefined size category comprises less than about 3 cm, about 3 cm to about 6 cm, or greater than about 6 cm.
140. The non-transitory computer-readable medium of clause 136, wherein the fixation modality selection is based on a bone-density indicator.
141. The non-transitory computer-readable medium of clause 136, wherein the fixation modality selection includes a screwless clamp for distal anchoring.
142. The non-transitory computer-readable medium of clause 136, wherein the system outputs a soft-tissue bridging module selection.
143. The non-transitory computer-readable medium of clause 136, wherein the system outputs a kit pick-list for sterile components.
144. The non-transitory computer-readable medium of clause 136, wherein the system outputs a fabrication specification for at least one patient-specific component.
145. The non-transitory computer-readable medium of clause 144, wherein the fabrication specification comprises a 3D printing file or a mold geometry.
146. The non-transitory computer-readable medium of clause 136, wherein the system outputs a warning indicator if the target distraction distance exceeds a threshold.
147. The non-transitory computer-readable medium of clause 136, wherein the system stores the recommended thoracic expansion construct in a patient record.
148. The non-transitory computer-readable medium of clause 136, wherein the system outputs a plan for re-exploration risk management using a temporary expander.
149. The non-transitory computer-readable medium of clause 136, wherein the system accepts intraoperative measurement updates and recomputes the target distraction distance.
150. The non-transitory computer-readable medium of clause 136, wherein the system outputs an estimated post-expansion thoracic cavity depth or volume.

A thoracic cavity expansion implant may include a spacer assembly configured to be positioned between a first thoracic cage segment and a second thoracic cage segment that are separated during a surgical operation, the spacer assembly defining a distraction distance that increases a thoracic cavity volume; and a fixation assembly configured to secure the spacer assembly to the first thoracic cage segment and the second thoracic cage segment to maintain the distraction distance. In an embodiment, the first thoracic cage segment and the second thoracic cage segment include a first sternal segment and a second sternal segment, respectively. Additionally, in one or more embodiments, the fixation assembly includes one or more fixation plates configured for attachment to an anterior surface of the thoracic cage segments. The fixation assembly may include bone screws, and/or a screwless fixation arrangement includes at least one of a clamp, a strap, a cable, a wire, or an adhesive interface. In some embodiments, the spacer assembly includes a monolithic spacer body, and in one or more embodiments, the monolithic spacer body may include a curable material contained by a mesh envelope. In various embodiments, the curable material includes polymethyl methacrylate (PMMA), methyl acrylate, methyl methacrylate, calcium phosphate cement, or ceramic composite cement. In an embodiment, the spacer assembly may be formed to have a curved profile corresponding to chest wall curvature at an implant site. Additionally, in one or more embodiments, the thoracic cavity expansion implant may further include rib-stabilizing sutures configured to stabilize an intercostal gap enlarged by the distraction distance. In some embodiments, the spacer assembly may include an antimicrobial surface treatment or an antibiotic-eluting material configured to reduce infection risk. In various embodiments, the fixation assembly may provide at least three fixation points on a first side of a sternotomy and at least two fixation points on a second side of the sternotomy. Furthermore, in some embodiments, the thoracic cavity expansion implant may also include a quick-release interface configured to permit removal or exchange of the spacer assembly without removing bone-attached hardware. Additionally, in one or more embodiments, the spacer assembly may include one or more radiopaque markers or may be configured to maintain the distraction distance during closure of soft tissue over the implant, and, in some embodiments, the distraction distance may be selected to be less than about 3 cm, from about 3 cm to about 6 cm, or greater than about 6 cm.

A modular thoracic cavity expansion system may include a base platform configured for fixation to a first sternal segment, a cap platform configured for fixation to a second sternal segment, a plurality of stackable spacer modules each defining a standardized module height and each comprising a central pass-through aperture, and a central retention rod configured to extend between the base platform and the cap platform through the central pass-through aperture of the plurality of stackable spacer modules and including a locking mechanism configured to compress the plurality of stackable spacer modules into a rigid load-bearing column that maintains a selected distraction distance between the first sternal segment and the second sternal segment. Additionally, in one or more embodiments, the standardized module height may be about 10 mm. In some embodiments, the base platform or the cap platform may include lateral flanges having fastener holes configured to receive bone screws, and the base platform or the cap platform may be configured for screwless fixation to a sternal segment. Furthermore, the cap platform may be configured to couple to the central retention rod via a threaded engagement, a bayonet lock, a cam lock, or a ratcheting collet. In various embodiments, the locking mechanism may include a threaded shaft portion of the central retention rod and a locking nut configured to thread onto the threaded shaft portion. Furthermore, in one or more embodiments, the base platform may include a polyaxial head configured to permit angulation of the central retention rod relative to the base platform. In one or more embodiments, the spacer modules may include keyed interlocking features on opposing faces configured to resist rotation of the spacer modules relative to one another about the central retention rod, and the spacer modules may also include surface textures configured to increase frictional resistance to shear displacement and/or a radiopaque marker indicating the standardized module height. The spacer modules may also be provided as a sterile kit including a plurality of different module heights. In some embodiments, the modular thoracic cavity expansion system also includes a soft-tissue bridging module having a reinforced central aperture configured to receive the central retention rod and lateral porous extensions configured for suturing soft tissue, and the soft-tissue bridging module may be configured to be retained by compressive force between adjacent spacer modules. Additionally, in one or more embodiments, the modular thoracic cavity expansion system further includes a distal anchoring clamp coupled to the base platform and configured to grip a xiphoid process or costal margin without a penetrating fastener, and the distal anchoring clamp may include opposed jaws and an actuator configured to generate clamping force. In various embodiments, the modular thoracic cavity expansion system may be configured for use with a transverse sternotomy or a clamshell incision. Furthermore, in some embodiments, the rigid load-bearing column may define a distraction distance selected from less than about 3 cm, about 3 cm to about 6 cm, and greater than about 6 cm. In one or more embodiments, the modular thoracic cavity expansion system also includes an antimicrobial coating on at least one of the spacer modules, the base platform, or the cap platform. In some embodiments, the spacer modules may be configured to be assembled intraoperatively to achieve a custom distraction distance.

A method for selecting a patient-specific thoracic expansion construct may include receiving recipient data comprising at least one of a recipient chest cavity volume or recipient thoracic geometry, receiving donor data comprising at least one of a donor lung volume or donor lung geometry, calculating a volumetric mismatch value based on the recipient data and the donor data, determining, using the volumetric mismatch value, a target distraction distance, and selecting, based on the target distraction distance, at least one of (i) a modular spacer module count, (ii) a monolithic spacer geometry, or (iii) a selection among predefined implant categories. In some embodiments, calculating the volumetric mismatch value may include subtracting the recipient chest cavity volume from the donor lung volume. In one or more embodiments, selecting among predefined implant categories may include selecting a small category less than about 3 cm, a medium category from about 3 cm to about 6 cm, or a large category greater than about 6 cm. Furthermore, in various embodiments, determining the target distraction distance may include applying a predictive expansion algorithm, and the predictive expansion algorithm may compute the target distraction distance as a function of the volumetric mismatch value and one or more thoracic geometry parameters. Additionally, in one or more embodiments, receiving recipient data may include receiving imaging-derived geometry from CT or MRI, and/or receiving donor data may include receiving imaging-derived donor lung geometry. In various embodiments, the recipient data may include at least one of height, weight, sex, chest size, or a fibrosis/scarring indicator, and the donor data may include at least one of donor height, donor sex, donor predicted total lung capacity, or a donor lung volume proxy. In some embodiments, the method may further include outputting a module count for spacer modules having a standardized height of about 10 mm, verifying the target distraction distance intraoperatively using a sterile gauge temporarily inserted into a sternotomy gap, and/or outputting a recommended fixation modality based on bone density indicators, and the fixation modality may be selected from screws, cerclage cables, straps, clamps, or adhesive interfaces. In various embodiments, the method may also include outputting a recommended soft-tissue closure strategy including use of a soft-tissue bridging module and/or outputting a curvature specification for the expansion construct. Additionally, in one or more embodiments, selecting a patient-specific thoracic expansion construct may include selecting a distal anchoring clamp when distal sternal bone quality is below a threshold and/or selecting a temporary or removable expander when a bleeding-risk indicator exceeds a threshold. In various embodiments, the method also includes generating a kit pick-list identifying sterile components to open for the procedure and/or storing the target distraction distance and the selected construct in a patient record. Furthermore, in some embodiments, the method is performed at least in part by a computing device.

A method for expanding a thoracic cavity during a surgical operation may include creating a sternotomy gap between a first sternal segment and a second sternal segment, separating the first sternal segment and the second sternal segment to increase a thoracic cavity volume, positioning a thoracic expansion implant in the sternotomy gap to establish a distraction distance, securing the thoracic expansion implant to the first sternal segment and the second sternal segment to maintain the distraction distance, and closing soft tissue over the thoracic expansion implant. In some embodiments, creating the sternotomy gap may include performing a transverse sternotomy and/or performing a clamshell incision. In various embodiments, securing may include selecting a screw fixation, a screwless fixation, or a hybrid fixation based on bone quality, and/or may include distributing load across osteopenic bone using an enlarged plate footprint or an adhesive-augmented interface. Additionally, in one or more embodiments, positioning the thoracic expansion implant comprises installing a monolithic cement spacer contained within mesh and/or assembling a modular column from stackable spacer modules on a central retention rod. In various embodiments, the method may also include placing rib-stabilizing sutures between ribs adjacent the sternotomy gap, implanting donor lungs or a donor heart after securing the thoracic expansion implant, installing a soft-tissue bridging module retained by the thoracic expansion implant and suturing soft tissue to porous lattice extensions of the soft-tissue bridging module, and/or engaging a distal anchoring clamp that grips a xiphoid process or costal margin without screws. Furthermore, in one or more embodiments, the method may also include selecting the distraction distance (i) based on recipient thoracic capacity and donor lung size prior to positioning the thoracic expansion implant, and/or (ii) intraoperatively using a sterile sizing gauge. Additionally, in some embodiments, the thoracic expansion implant may include an antimicrobial surface treatment or an antibiotic-eluting material. In one or more embodiments, the distraction distance may be less than about 3 cm, from about 3 cm to about 6 cm, or greater than about 6 cm. In various embodiments, the method may further include using a temporary expander in the sternotomy gap and removing the temporary expander prior to final closure, removing or exchanging the thoracic expansion implant via a quick-release interface during a re-exploration procedure, applying a negative-pressure dressing over a closed incision following closure, and/or placing a biologic scaffold over the thoracic expansion implant prior to closure.

A soft-tissue bridging module for use with a thoracic expansion construct may include a flexible sheet body having a medial portion and a lateral extension, and a reinforced central aperture in the medial portion configured to receive a retention rod or a fastener of the thoracic expansion construct, and further, the flexible sheet body may be configured to be mechanically retained by the thoracic expansion construct and the lateral extension may include a porous lattice configured to receive sutures for attaching soft tissue. In various embodiments, reinforced central aperture may include a grommet formed from titanium or polyether ether ketone (PEEK). Additionally, in one or more embodiments, the porous lattice may include a bioabsorbable polymer mesh configured to promote tissue ingrowth, a permanent synthetic mesh configured to provide long-term reinforcement, and/or a lattice pattern selected from a grid, a diamond mesh, or a radial spoke pattern. Furthermore, in at least one embodiment, the flexible sheet body may include an acellular dermal matrix or a composite of biologic and synthetic layers, an antibiotic elution, and/or a low-biofilm surface texture. In various embodiments, the lateral extension may include suture tabs positioned to align with pectoralis, fascial, or dermal edges. In one or more embodiments, the soft-tissue bridging module may further be configured to be sandwiched between adjacent spacer modules of a modular column, configured to be secured to a fixation plate of a cement spacer construct, and/or configured to be trimmed intraoperatively while preserving suture lattice integrity. Additionally, in some embodiments, the soft-tissue bridging module may also include radiopaque markers and/or an integrated barrier layer configured to reduce adhesions. In at least one embodiment, the soft-tissue bridging module may be provided sterile-packaged and labeled for a predefined distraction category.

A distal anchoring clamp for a thoracic expansion construct may include a first jaw and a second jaw configured to engage a xiphoid process or costal margin region, an actuator configured to generate clamping force between the first jaw and the second jaw without a penetrating bone fastener, and a construct interface configured to couple the clamp to a thoracic expansion implant to resist migration of the implant. In one or more embodiments, the first jaw and the second jaw may include friction pads or textured surfaces configured to increase resistance to slippage. Additionally, in various embodiments, the actuator may include a screw mechanism or a cam-lock mechanism. In some embodiments, the construct interface may include a plate arm having fastener holes for coupling to a base platform or fixation plate. Furthermore, in one or more embodiments, the clamp may include a load-distributing jaw footprint configured for osteopenic anatomy, an adjustable jaw spacing to accommodate variable anatomy, radiopaque markers, an antimicrobial coating, and/or a breakaway feature configured to limit clamping force and reduce tissue injury. In at least one embodiment, the claim may be configured to engage costal cartilage, configured to engage tendinous or fascial tissue adjacent the xiphoid process, configured for tool-less release after implantation, and/or configured to cooperate with cerclage cables or straps. In some embodiments, the clamp may be provided as part of a sterile kit with spacer modules.

A temporary thoracic expansion system may include an expandable element configured to be positioned within a sternotomy gap to maintain a temporary distraction distance, a fluid port configured to permit adjustment of the expandable element after placement, and a fixation arrangement configured for rapid release, and the expandable element may be configured to be collapsed and withdrawn to permit rapid chest closure or re-exploration. Additionally, in one or more embodiments, the expandable element may include an inflatable bladder, a pressure-limiting feature configured to limit distraction force, radiopaque markers, an antimicrobial surface treatment, an internal baffle configured to reduce migration, and/or a shape selected to match a sternal curvature. In various embodiments, the expandable element may be configured to be removed through a limited incision. In at least one embodiment, the fluid port may be configured to remain accessible from an exterior of a patient and/or configured for connection to a sterile syringe or pump. Furthermore, in one or more embodiments, the fixation arrangement may include removable clips and/or biodegradable sutures. In some embodiments, the temporary thoracic expansion system may be configured to be replaced with a permanent spacer construct after a bleeding-risk period. Additionally, in one or more embodiments, the temporary thoracic expansion system further includes a quick-release coupler configured to connect the temporary system to a base platform or fixation plate. In at least one embodiment, the temporary distraction distance may be adjustable between about 1 cm and about 8 cm.

A surgical kit for thoracic cavity expansion may include a plurality of sterile-packaged thoracic expansion constructs corresponding to discrete distraction distance ranges and at least one sterile sizing instrument configured to be temporarily inserted into a sternotomy gap to verify a distraction distance prior to selecting one of the thoracic expansion constructs. In some embodiments, the discrete distraction distance ranges may include a first range less than about 3 cm, a second range from about 3 cm to about 6 cm, and a third range greater than about 6 cm. In one or more embodiments, at least one of the thoracic expansion constructs may include a modular system including spacer modules and a central retention rod, and at least one of the thoracic expansion constructs may include a cement spacer contained within mesh and associated fixation plates. Additionally, in at least one embodiment, the sizing instrument may include a stepped gauge corresponding to standardized module increments. Furthermore, in one or more embodiments, the surgical kit may also include a plurality of soft-tissue bridging modules configured for use with the thoracic expansion constructs, a distal anchoring clamp configured to grip a xiphoid process or costal margin, a selection guide mapping distraction distance to module count, cerclage cables or straps for screwless fixation, an antimicrobial irrigation or coating application component, a temporary inflatable expander, a quick-release interface component for spacer exchange, spacer modules of multiple thicknesses to provide fine adjustment, and/or a sterile template for matching curvature to a patient anatomy. Additionally, in at least one embodiment, each sterile package may include machine-readable labeling encoding distraction range and curvature profile.

A non-transitory computer-readable medium may store instructions that, when executed by one or more processors, cause a system to receive recipient thoracic geometry data and recipient thoracic volume data, receive donor lung geometry data and donor lung volume data, compute a volumetric mismatch value, compute a target distraction distance based on the volumetric mismatch value, and output a recommended thoracic expansion construct including at least one of a module count, a predefined size category, a curvature specification, or a fixation modality selection. In some embodiments, the recipient thoracic geometry data may include CT-derived geometry. Additionally, in one or more embodiments, the system may output a module count for spacer modules of about 10 mm height, a soft-tissue bridging module selection, a kit pick-list for sterile components, a fabrication specification for at least one patient-specific component, a warning indicator if the target distraction distance exceeds a threshold, a plan for re-exploration risk management using a temporary expander, and/or an estimated post-expansion thoracic cavity depth or volume. In at least one embodiment, the fabrication specification may include a 3D printing file or a mold geometry. Furthermore, in at least one embodiment, the predefined size category may be less than about 3 cm, about 3 cm to about 6 cm, or greater than about 6 cm. In some embodiments, the fixation modality selection may be based on a bone-density indicator and/or may include a screwless clamp for distal anchoring. In various embodiments, the system may store the recommended thoracic expansion construct in a patient record. Additionally, in at least one embodiment, the system may accept intraoperative measurement updates and recomputes the target distraction distance.

One or more embodiments may further be described in view of the following clauses:
1. A thoracic cavity expansion device for installation in a thoracic cage during a surgical procedure, the device comprising:
a molded body comprising:
   a bone cement; and
   a mesh envelope substantially surrounding the bone cement, the mesh envelope constraining the bone cement to define a shape of the device; and
one or more fasteners arranged to secure the molded body to an upper sternal portion and to a lower sternal portion of the thoracic cage;
wherein the device spans a sternotomy gap between the upper sternal portion and the lower sternal portion and maintains a separation between the upper and lower sternal portions that increases a post-surgical cavity volume of the thoracic cage relative to a pre-surgical cavity volume.
2. The device of clause 1, further comprising at least one fixation plate mechanically coupled to the molded body.
3. The device of clause 2, wherein the one or more fasteners secure the at least one fixation plate to at least one of the upper sternal portion or the lower sternal portion.
4. The device of clause 2, wherein the at least one fixation plate comprises titanium.
5. The device of clause 1, wherein the bone cement comprises methyl acrylate.
6. The device of clause 1, wherein the molded body is formed at a curve.
7. The device of clause 1, wherein at least one of a length, a thickness, a width, a shape, or a size of the device is determined by an algorithmic model.
8. The device of clause 1, wherein a device length, a thickness, a width, a shape, or a size is based on patient parameters comprising at least one of a recipient height, the pre-surgical cavity volume, or a donated organ volume.
9. The device of clause 1, wherein the molded body has a length of about 3 cm to about 5 cm and a width of about 1 cm to about 2 cm.
10. The device of clause 1, wherein the one or more fasteners provide at least three points of attachment to the upper sternal portion and at least two points of attachment to the lower sternal portion.
11. The device of clause 1, wherein the device is installed via a sternotomy in the thoracic cage.
12. A surgical method for expanding a thoracic cage during a surgical procedure, comprising:
cutting through a thoracic cage to create an upper thoracic cage portion, a lower thoracic cage portion, an upper sternal portion, and a lower sternal portion;
separating the first thoracic cage portion and the second thoracic cage portion to open the thoracic cage;
installing, between the first thoracic cage portion and the second thoracic cage portion, a device that holds the first thoracic cage portion and the second thoracic cage portion apart from each other to expand a thoracic cavity of the thoracic cage, wherein the device comprises:
   bone cement positioned between the first sternal portion and the second sternal portion;
   one or more fixation plates; and
   one or more fasteners; and
fastening the one or more fixation plates to the bone cement, the first sternal portion, and the second sternal portion with the one or more fasteners;
wherein a post-surgical cavity volume of the thoracic cage is larger relative to a pre-surgical cavity volume of the thoracic cage.
13. The surgical method of clause 12, further comprising:
installing sutures in the thoracic cage to stabilize ribs, the sutures positioned between the first thoracic cage portion and the second thoracic cage portion.
14. The surgical method of clause 12, further comprising:
advancing a pectoralis muscle of the thoracic cage.
15. The surgical method of clause 12, wherein the advancement of the pectoralis muscle is at least in part due to the expansion of the thoracic cage from the pre-surgical cavity volume to the post-surgical cavity volume
16. The surgical method of clause 12, further comprising:
closing a cut in the thoracic cage made during the cutting of the thoracic cage.
17. The surgical method of clause 12, wherein the cutting comprises a horizontal sternotomy substantially perpendicular to a sternum.
18. The surgical method of clause 12, wherein the cutting comprises a median sternotomy substantially parallel to or along a sternum.
19. The surgical method of clause 12, wherein the device further comprises a mesh envelope substantially surrounding the bone cement and providing a shape for the device by containing the bone cement.
20. The surgical method of clause 12, further comprising:
   forming the device at a curve relative to the thoracic cage.
21. A thoracic cavity expansion device for installation in a thoracic cage during a surgical procedure, the device comprising:
a molded body comprising:
   a bone cement; and
   a mesh envelope substantially surrounding the bone cement, the mesh envelope constraining the bone cement to define a shape of the device; and
one or more fasteners arranged to secure the molded body to an upper sternal portion and to a lower sternal portion of the thoracic cage;
wherein the device spans a sternotomy gap between the upper sternal portion and the lower sternal portion and maintains a separation between the upper and lower sternal portions that increases a post-surgical cavity volume of the thoracic cage relative to a pre-surgical cavity volume.
22. The device of clause 21, further comprising at least one fixation plate mechanically coupled to the molded body.
23. The device of clause 22, wherein the one or more fasteners secure the at least one fixation plate to at least one of the upper sternal portion or the lower sternal portion.
24. The device of any of clauses 22 or 23, wherein the at least one fixation plate comprises titanium.
25. The device of any of clauses 21-24, wherein the bone cement comprises methyl acrylate.
26. The device of any of clauses 21-25, wherein the molded body is formed at a curve.
27. The device of any of clauses 21-26, wherein at least one of a length, a thickness, a width, a shape, or a size of the device is determined by an algorithmic model.
28. The device of any of clauses 21-27, wherein a device length, a thickness, a width, a shape, or a size is based on patient parameters comprising at least one of a recipient height, the pre-surgical cavity volume, or a donated organ volume.
29. The device of any of clauses 21-28, wherein the molded body has a length of about 3 cm to about 5 cm and a width of about 1 cm to about 2 cm.
30. The device of any of clauses 21-29, wherein the one or more fasteners provide at least three points of attachment to the upper sternal portion and at least two points of attachment to the lower sternal portion.
31. The device of any of clauses 21-30, wherein the device is installed via a sternotomy in the thoracic cage.
32. A surgical method for expanding a thoracic cage during a surgical procedure, comprising:
cutting through a thoracic cage to create an upper thoracic cage portion, a lower thoracic cage portion, an upper sternal portion, and a lower sternal portion;
separating the first thoracic cage portion and the second thoracic cage portion to open the thoracic cage;
installing, between the first thoracic cage portion and the second thoracic cage portion, a device that holds the first thoracic cage portion and the second thoracic cage portion apart from each other to expand a thoracic cavity of the thoracic cage, wherein the device comprises:
   bone cement positioned between the first sternal portion and the second sternal portion;
   one or more fixation plates; and
   one or more fasteners; and
fastening the one or more fixation plates to the bone cement, the first sternal portion, and the second sternal portion with the one or more fasteners;
wherein a post-surgical cavity volume of the thoracic cage is larger relative to a pre-surgical cavity volume of the thoracic cage.
33. The surgical method of clause 32, further comprising:
installing sutures in the thoracic cage to stabilize ribs, the sutures positioned between the first thoracic cage portion and the second thoracic cage portion.
34. The surgical method of any of clauses 32 or 33, further comprising:
advancing a pectoralis muscle of the thoracic cage.
35. The surgical method of any of clauses 32-34, wherein the advancement of the pectoralis muscle is at least in part due to the expansion of the thoracic cage from the pre-surgical cavity volume to the post-surgical cavity volume
36. The surgical method of any of clauses 32-35, further comprising:
closing a cut in the thoracic cage made during the cutting of the thoracic cage.
37. The surgical method of any of clauses 32-36, wherein the cutting comprises a horizontal sternotomy substantially perpendicular to a sternum.
38. The surgical method of any of clauses 32-37, wherein the cutting comprises a median sternotomy substantially parallel to or along a sternum.
39. The surgical method of any of clauses 32-38, wherein the device further comprises a mesh envelope substantially surrounding the bone cement and providing a shape for the device by containing the bone cement.
40. The surgical method of any of clauses 32-39, further comprising:
   forming the device at a curve relative to the thoracic cage.

Although the technology herein has been described with reference to particular embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

What is claimed is:

1. A thoracic cavity expansion device for installation in a thoracic cage during a surgical procedure, the device comprising:
a molded body comprising:
a bone cement; and
a mesh envelope substantially surrounding the bone cement, the mesh envelope constraining the bone cement to define a shape of the device; and
one or more fasteners arranged to secure the molded body to an upper sternal portion and to a lower sternal portion of the thoracic cage;
wherein the device spans a sternotomy gap between the upper sternal portion and the lower sternal portion and maintains a separation between the upper and lower sternal portions that increases a post-surgical cavity volume of the thoracic cage relative to a pre-surgical cavity volume.

2. The device of claim 1, further comprising at least one fixation plate mechanically coupled to the molded body.

3. The device of claim 2, wherein the one or more fasteners secure the at least one fixation plate to at least one of the upper sternal portion or the lower sternal portion.

4. The device of claim 2, wherein the at least one fixation plate comprises titanium.

5. The device of claim 1, wherein the bone cement comprises methyl acrylate.

6. The device of claim 1, wherein the molded body is formed at a curve.

7. The device of claim 1, wherein at least one of a length, a thickness, a width, a shape, or a size of the device is determined by an algorithmic model.

8. The device of claim 1, wherein a device length, a thickness, a width, a shape, or a size is based on patient parameters comprising at least one of a recipient height, the pre-surgical cavity volume, or a donated organ volume.

9. The device of claim 1, wherein the molded body has a length of about 3 cm to about 5 cm and a width of about 1 cm to about 2 cm.

10. The device of claim 1, wherein the one or more fasteners provide at least three points of attachment to the upper sternal portion and at least two points of attachment to the lower sternal portion.

11. The device of claim 1, wherein the device is installed via a sternotomy in the thoracic cage.

12. A surgical method for expanding a thoracic cage during a surgical procedure, comprising:
cutting through a thoracic cage to create an upper thoracic cage portion, a lower thoracic cage portion, an upper sternal portion, and a lower sternal portion;
separating the first thoracic cage portion and the second thoracic cage portion to open the thoracic cage;
installing, between the first thoracic cage portion and the second thoracic cage portion, a device that holds the first thoracic cage portion and the second thoracic cage portion apart from each other to expand a thoracic cavity of the thoracic cage, wherein the device comprises:
bone cement positioned between the first sternal portion and the second sternal portion;
one or more fixation plates; and
one or more fasteners; and
fastening the one or more fixation plates to the bone cement, the first sternal portion, and the second sternal portion with the one or more fasteners;
wherein a post-surgical cavity volume of the thoracic cage is larger relative to a pre-surgical cavity volume of the thoracic cage.

13. The surgical method of claim 12, further comprising:
installing sutures in the thoracic cage to stabilize ribs, the sutures positioned between the first thoracic cage portion and the second thoracic cage portion.

14. The surgical method of claim 12, further comprising:
advancing a pectoralis muscle of the thoracic cage.

15. The surgical method of claim 12, wherein the advancement of the pectoralis muscle is at least in part due to the expansion of the thoracic cage from the pre-surgical cavity volume to the post-surgical cavity volume

16. The surgical method of claim 12, further comprising:
closing a cut in the thoracic cage made during the cutting of the thoracic cage.

17. The surgical method of claim 12, wherein the cutting comprises a horizontal sternotomy substantially perpendicular to a sternum.

18. The surgical method of claim 12, wherein the cutting comprises a median sternotomy substantially parallel to or along a sternum.

19. The surgical method of claim 12, wherein the device further comprises a mesh envelope substantially surrounding the bone cement and providing a shape for the device by containing the bone cement.

20. The surgical method of claim 12, further comprising:
forming the device at a curve relative to the thoracic cage.
